(19) United States
(12) Patent Application Publication
(10) Pub. No.: US 2022/0125623 A1
(43) Pub. Date: Apr. 28, 2022

(54) Heating Device

(71) Applicant: Julie Anne Labrie Lancaster, Skowhegan, ME (US)

(72) Inventor: Julie Anne Labrie Lancaster, Skowhegan, ME (US)

(21) Appl. No.: 17/507,875

(22) Filed: Oct. 22, 2021

Related U.S. Application Data
(60) Provisional application No. 63/105,372, filed on Oct. 26, 2020.

(51) Int. Cl. A61F 7/00

(52) U.S. Cl. CPC A61F 7/007; A61F 2007/0071; A61F 2007/0244; A61F 2007/0029; A61F 2007/0039; A61F 2007/008

(57) ABSTRACT

A heating device that is adapted to fit on a limb of a body to provide heat to said limb, the heating device being a wearable and portable device.

### BACKGROUND INFORMATION

### Field of the Invention

The invention relates to heating devices, and in particular heating devices that fit on limbs of a body such as arms and legs.

### Discussion of Prior Art

Heat is known to provide many benefits, from increasing one's comfort level to providing therapeutic benefits in a number of different situations. Means for providing such heat come in a number of forms, including both mechanical and electrical heating devices as well as topical ointments. Heating devices in particular include such items as heating pads, hot water bottles, and heat wraps.

Such devices are often useful when a user is able to sit and be still, and in many cases when the user is able to be near a power source while the device is in use. However, many instances exist where a user is not able to sit and idly apply the heat beside a power source. For example, heat therapy is often needed on a frequent or on-going basis while a user/wearer is performing routine tasks. In other cases, heat provides a great deal of comfort as someone moves about in cold weather conditions.

What is needed, therefore, is a heating device that fits securely on limb, such as an arm or a leg and that holds a secure position as the wearer moves about over an extended period of time.

### BRIEF SUMMARY OF THE INVENTION

The invention is a portable and wearable heating device that securely wraps around a limb, such as an arm or a leg, and that is able to maintain the desired position as the user moves about. More specifically, the device fits securely in place around the limb so that a user may be active while using the device, the device generally providing heat to the majority of the area being covered.

In a primary embodiment the device is approximately the length of the limb and wraps securely around the entirely of the limb. The heating source includes electrically powered wires, the wires largely extending through the length of the device and being connected to one or more portable batteries that are small enough to be conveniently worn along with the device itself.

### BRIEF DESCRIPTION OF THE DRAWINGS

The present invention is described with reference to the accompanying drawings. In the drawings, like reference numbers indicate identical or functionally similar elements. The drawings are not drawn to scale.

FIG. 1 is a top plan view of an embodiment of the heating device according to the invention.

FIG. 2 is top plan view of the device in an open configuration showing two battery packs and two control units.

FIG. 3 is a top plan view on an internal layer of device showing the heating element.

FIG. 4 is a top view of the interior of the heating device.

FIG. 5 a view of a portion of a heating element on an end of the heating device.

FIG. 6 is a close-up view of an edge of the heating device in an open position.

### DETAILED DESCRIPTION OF THE INVENTION

The present invention will now be described more fully in detail with reference to the accompanying drawings, in which the preferred embodiments of the invention are shown. This invention should not, however, be construed as limited to the embodiments set forth herein; rather, they are provided so that this disclosure will be complete and will fully convey the scope of the invention to those skilled in the art.

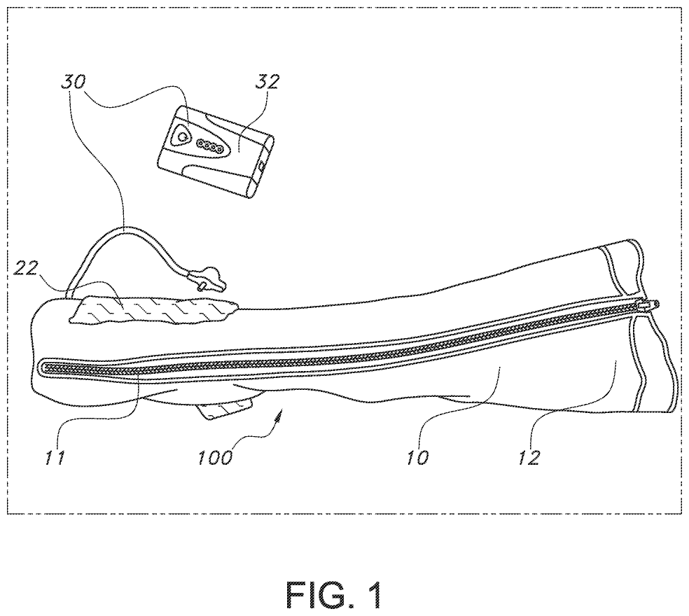

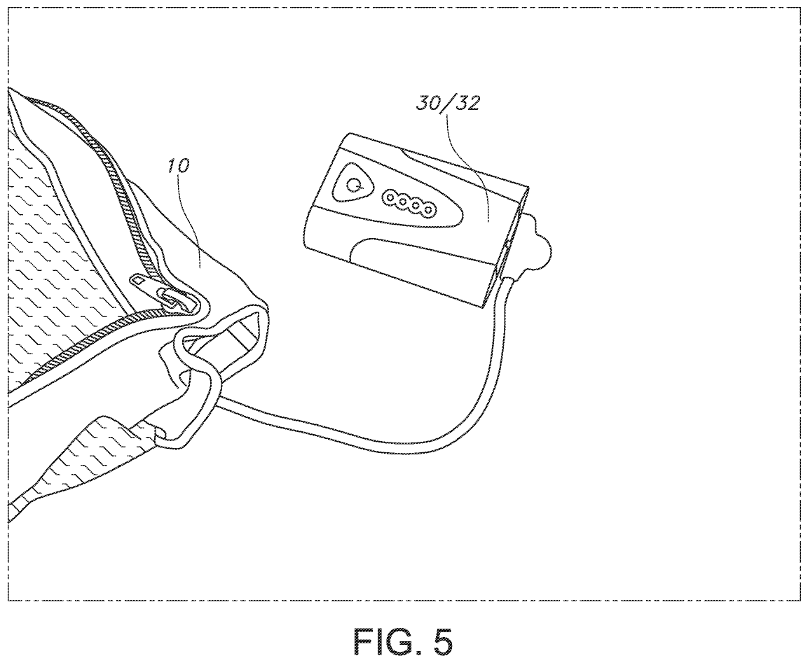

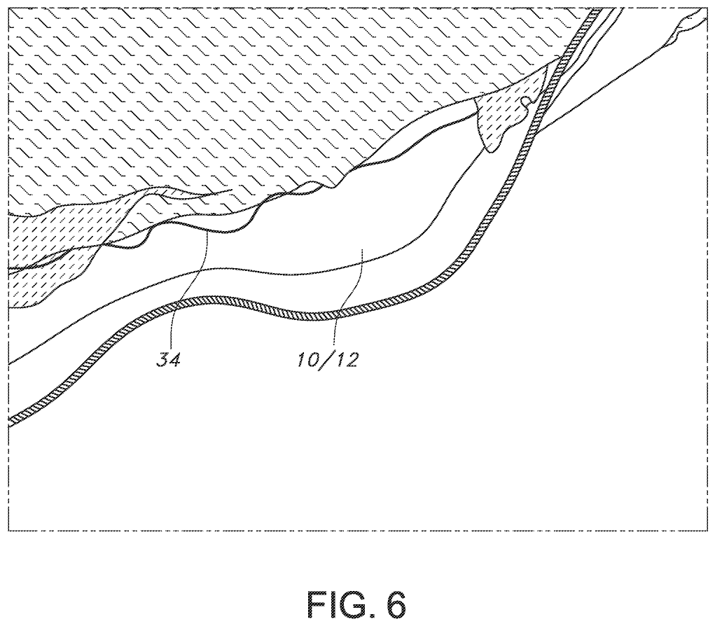

FIGS. 1-6 illustrate a heating device 100 according to the invention that includes a covering 10 that fits securely around a limb of a body, such as an arm or a leg, and that includes a heating element 30 that provides heat to at least a portion of the body part.

The covering 10 is sized and shaped to fit to a particular limb, and is likely to be manufactured in a number of sizes to conform to limbs that are larger and smaller as a snug and secure fit against the body part is important to the overall function. The covering 10 also has a securing means 11, such as a zipper, laces, or an elastic material. The heating element 30 is likely to run throughout the covering 10 so as to provide a consistent and relatively uniform amount of heat over and around the entire area of the body part that is covered by the device. If the heating element is electrically powered it is likely that a rechargeable battery pack is attached to the device 100/30 and located outside the covering 30 as a power source for the portion of the heating element 30 inside the covering 10.

For example, the covering 10 may be a sheet of fabric material in the form of a sleeve that wraps around a user's arm and provides warmth, either to increase a wearer's comfort on a cold day or to aid someone who needs or benefits from a therapeutic application of heat to a particular area of the body. This disclosure largely discusses the invention in terms of its use as a sleeve to be worn on a person's arm, however, it is understood that this is only an example and other designs and uses are also possible. For example, the heating device may be constructed to fit on a person's leg and/or be adapted to be worn by pets.

The covering 10 may also include a number of layers. For example, there may be an outer layer 12, that is an outer surface to the cover, that is preferably a resilient member that is able to handle the wear and tear of being worn frequently while a wearer moves about and engages in normal day-to-day activities, such as, for example, wool, polyester, and/or cotton. And there may be inner layers that may include batting fabric 14 and/or a layer of a soft fabric 16 that is comfortable against the wearer's skin such as cotton, the soft fabric 16 being an inner surface of the covering 10.

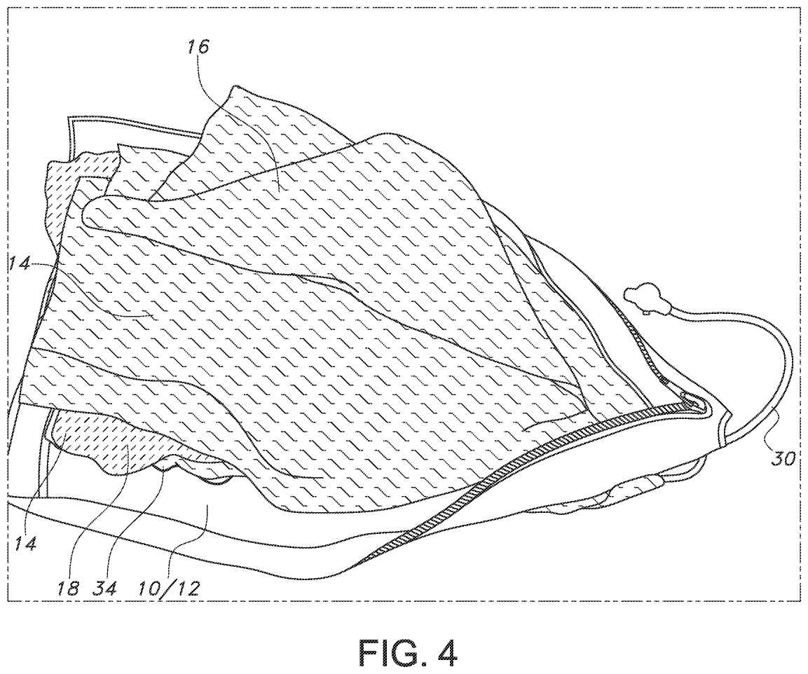

The embodiment shown in FIGS. 4-6, for example, includes, in order, the outer layer 12, the heating element 30, a first layer of batting 14, a layer of a plastic 18, a second layer of batting fabric 14, and then the soft fabric 16. The soft fabric may be made of a number of conventional materials such as cotton, wool, and/or polyester. The plastic layer may be made of any plastic that is relatively resistant to heat, such as, for example, Polytetrafluoroethylene (PTFE) or synthetic polymers composed of polyamides such as nylon.

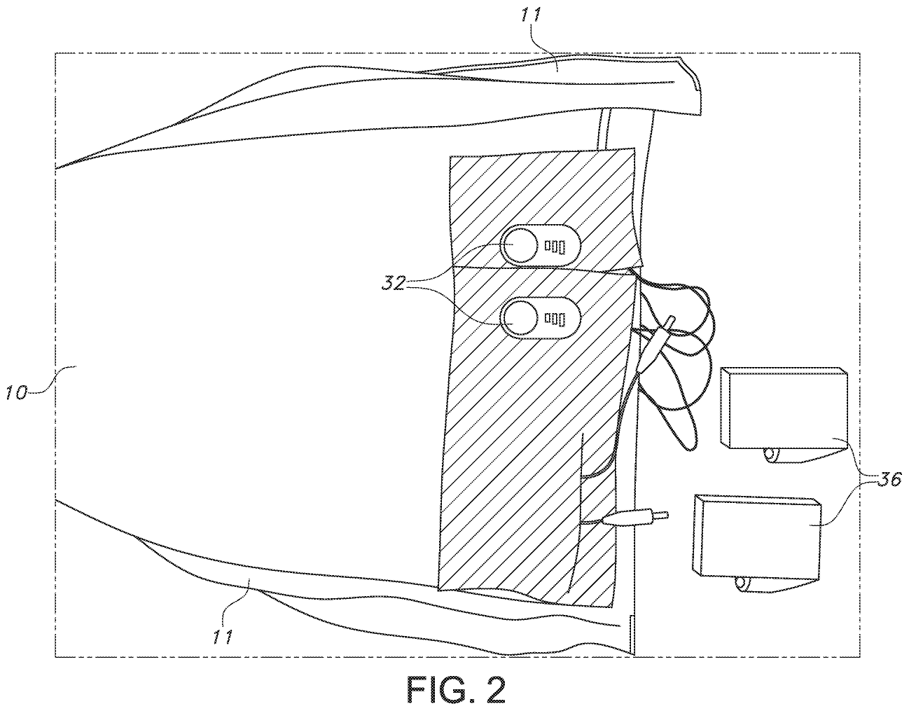

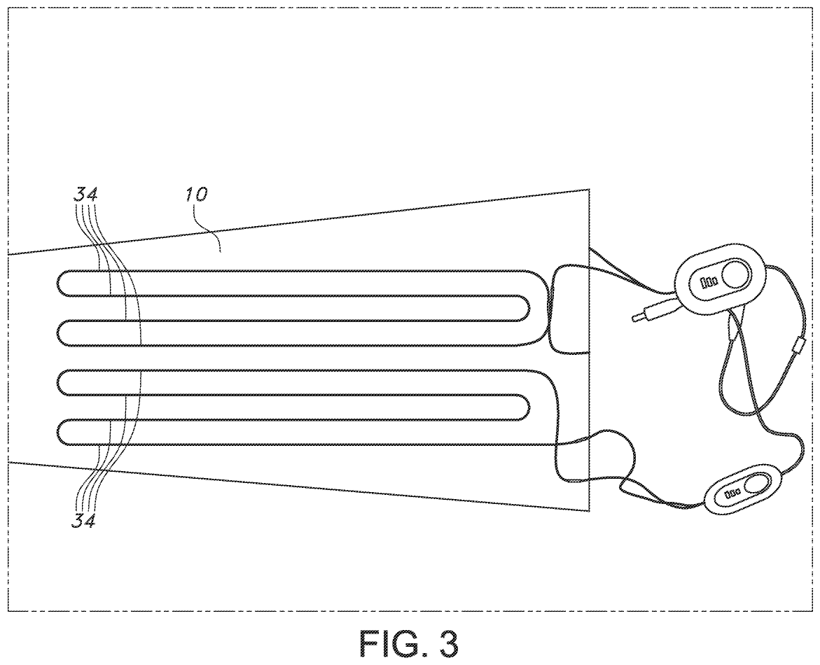

In another embodiment shown in FIGS. 2 and 3 there is an outer layer 12 and inner layer 16, each of which may be made of nylon, spandex and/or a combination of nylon and spandex, with a middle layer 14 that is made of cotton.

The covering 10 may be openable and closable, for example by including a zipper 11 or ties. Alternatively, the covering 10 may also have a degree of elasticity such that it is able to expand around a user's arm yet still fit securely in position, made of an elastic or stretch fabric lycra, elastane, and/or spandex. The covering 10 may also be in the form of a conventional compression sleeve for additional therapeutic benefits.

The embodiment shown in the Figures illustrate the use of a zipper 11 to close and secure the device around the limb. In this example, the device 100 may either be custom sized to fit a particular user, or it may be made in a variety of sizes such that each wearer may pick the size that is best suited for them. The closures may also be made in an adjustable manner, for example, by using hook and loop fasters or a lacing configuration.

There are a number of suitable ways to configure the heating element 30. In the embodiment shown the heating elements include one or two battery packs 36 and one or two control units 32 that heat one or more wires 34 that extend from the unit 32 into the covering 10. As previously noted, in the main embodiment the wires are evenly and uniform distributed to deliver heat to the majority of the limb covered by the device. However, the wire distribution may also be customized to target a specific area, for example, to provide therapeutic heat to a particular area of a user's arm.

There are a number of conventional battery powered heating wires that may be used for this embodiment, such as those that are conventionally used in heated clothing. For example, wire made of Nichrome, which is also known as NiCr, nickel-chromium or chromium-nickel and is a family of alloys of nickel, chromium, and often iron. Similarly there are a number of conventional battery packs that may be suitable. For example, lithium ion rechargeable batteries, such as the rechargeable 7.4V 2200 milliampere/hour (“MAH”)/3000 MAH Li-ion Batteries made by SUN WILL.

The battery pack and control unit may extend out of a portion of the covering near the user's wrist, however, it may also out the top of the covering, which would be at a location near the user's shoulder. An outer pouch 22 may be added to the outer surface of the covering 10 to hold the battery back and control unit 32. Other conventional non-electric heating elements are also suitable.

The control unit 32 is a simple conventional control that turns the unit on or off by controlling the flow of electricity from the battery to the wire. The control unit 32 may also be used to control the amount of electricity that passes from the battery to the wires to control the level of heat that is applied to the user; for example, based on the battery described above a low heat may be in the range of 100-110 degrees Fahrenheit, medium heat may be in the range of 120-130 degrees Fahrenheit, and high heat may be in the range of 140-150 degrees Fahrenheit. There are a number of conventional controls that may be used for this purpose, and those skilled in the art are able to choose the proper switch for the intended power source and heating wires, for example, the DC 3.7V-12V thermostat temperature controller timing switch manufactured by YFLZBY or the 5-speed reduction cycle temperature control switch manufactured by JECTSE.

Non-electric heating elements may also be suitable. For example, microwavable heating pads that are typically made out of a thick insulative fabric such as flannel and filled with grains such as rice, wheat, or flax seed.

It is understood that the embodiments described herein are merely illustrative of the present invention. Variations in the construction of the inventive heating device may be contemplated by one skilled in the art without limiting the intended scope of the invention herein disclosed and as defined by the following claims.

What is claimed is:

1. A heating device that is adapted to fit on or over a limb, the heating device comprising:
a covering that fits securely around the limb, the covering having an inner layer and an outer layer;
at least one heating element positioned between the inner layer and outer layer, the heating element adapted to provide heat to the limb.

2. The heating device of claim 1, wherein the heating element includes one or more wires that extend between the inner layer and outer layer.

3. The heating device of claim 2, wherein the covering includes a first layer of batting fabric that is next to a layer of plastic, the layer of plastic next to a second layer of batting fabric, each of the first layer of batting fabric, the layer of plastic, and the second layer of batting fabric located between the inner layer and outer layer.

4. The heating device of claim 2, wherein the heating element includes one or more battery packs that are connected to the one or more wires, the battery pack configured to provide electricity to the one or more wires to provide heat inside the covering.

5. The heating device of claim 4, the heating element including a control unit that controls the flow of electricity from the one or more battery pack to the one or more wires.

6. The device of claim 4 wherein the one or more battery pack is a rechargeable battery pack.
